(19) United States
(12) Patent Application Publication
(10) Pub. No.: US 2020/0093630 A1
(43) Pub. Date: Mar. 26, 2020

(54) ADJUSTABLE NECK COLLAR

(71) Applicant: YU-CHIEN WANG, Taichung City (TW)

(72) Inventor: YU-CHIEN WANG, Taichung City (TW)

(21) Appl. No.: 16/142,925

(22) Filed: Sep. 26, 2018

(51) Int. Cl. A61F 5/055

(52) U.S. Cl. CPC A61F 5/055

(57) ABSTRACT

An adjustable neck collar includes a neck collar main body, including a neck sleeve and a chin support; a barrel body has a first engaging toothed portion, an axle member and a reel member are axially movably pivoted to the barrel body and has a second engaging toothed portion, the reel member has a third engaging toothed portion detachably engaged with the first engaging toothed portion, the axle member is configured to drive the reel member to detach from the first engaging toothed portion, the second engaging toothed portion is detachably engaged with the third engaging toothed portion, a spring abuts against the barrel body and the reel member, at least one fastening member is disposed through the reel member and connected to the chin support.

### BACKGROUND OF THE INVENTION

### Field of the Invention

The present invention relates to an adjustable neck collar.

### Description of the Prior Art

Generally, the injured needs to wear medical protective equipment, for example, a neck collar, a waist belt, a knee pad or an orthopedic footwear, to protect injured body part. To meet a user's need, the neck collar usually provides height adjusting feature, and the user can adjust the height of an upper edge of the neck collar to support a front edge of his/her chin so that the user can look forward at eyelevel.

However, this type of conventional reel device includes a hook-and-loop fastener which is disposed on a protective gear, the hook-and-loop fastener can positionably hook a rope, the hook-and-loop fastener is pulled to drive the rope to move upward or downward and positionably hook the rope. However, the hook-and-loop fastener such as a Velcro can be easily pulled open and unable to tightly hook the rope; therefore, a force of the hook-and-loop fastener hooking the rope is not great enough, the rope may slide or fall off relative to the hook-and-loop fastener, and the protective gear is unable to tightly wrap on a body part.

The present invention has arisen to mitigate and/or obviate the afore-described disadvantages.

### SUMMARY OF THE INVENTION

The major object of the present invention is to provide an adjustable neck collar, which has a simple structure and is convenient for releasing and wrapping a line quickly.

To achieve the above and other objects, an adjustable neck collar is provided, including a neck collar main body, including a neck sleeve, a chin support which is movably connected to the neck sleeve, two connecting members and two position-restricting members, each of two opposite sides of the neck sleeve having a slant guide slot, each of the two connecting members being slidably disposed in one of the slant guide slots, the two position-restricting members being respectively disposed on the two opposite sides of the neck sleeve; a reel device, disposed on the neck sleeve, the reel device including a barrel body, an axle member, a reel member, a spring and at least one fastening member, a circumferential wall of the barrel body having a first engaging toothed portion, the axle member being axially movably pivoted to the barrel body, the axle member having a second engaging toothed portion, the reel member being axially movably pivoted to the barrel body, the reel member having a third engaging toothed portion, the third engaging toothed portion being detachably engaged with the first engaging toothed portion, the axle member being configured to drive the reel member to axially detach from the first engaging toothed portion, the second engaging toothed portion being detachably engaged with the third engaging toothed portion, the spring abutting against and between the barrel body and the reel member, the at least one fastening member being disposed through the reel member, the at least one fastening member being connected to the chin support, each of two end portions of the at least one fastening member being movably wrapped on one of the two position-restricting members and connected to one of the two connecting members.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of a preferred embodiment of the present invention;

FIG. 2 is a stereogram of a reel device of the preferred embodiment of the present invention;

FIG. 3 is a breakdown view of the reel device of the preferred embodiment of the present invention;

FIG. 4 is a cross-sectional view, taken along line A-A of FIG. 3;

FIG. 5 is a partially cross-sectional view of the reel device of the preferred embodiment of the present invention;

FIGS. 6 and 7 are partial views showing rope wrapping state of the reel device of the preferred embodiment of the present invention;

FIG. 8 is a partially enlarged view showing a third engaging toothed portion engaged with a first engaging toothed portion of the preferred embodiment of the present invention;

FIG. 9 is a partially enlarged view showing a second engaging toothed portion abutting against a third engaging toothed portion of the preferred embodiment of the present invention; and

FIG. 10 is a partially enlarged view showing a third engaging toothed portion moving to a tooth groove of the preferred embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

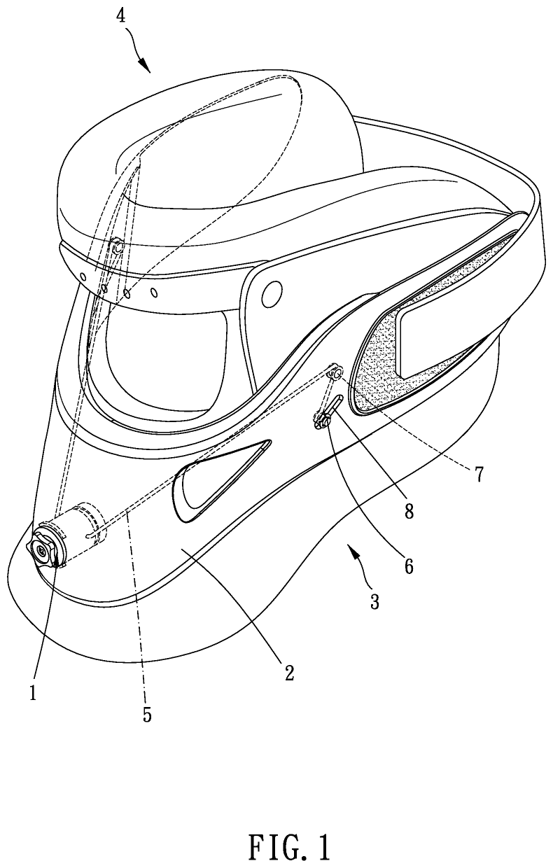

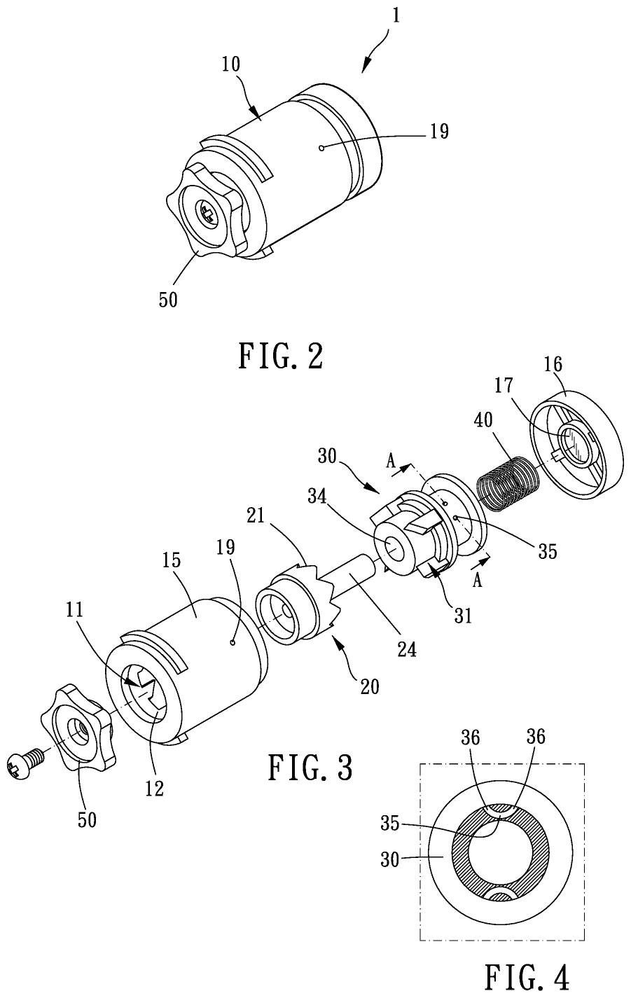

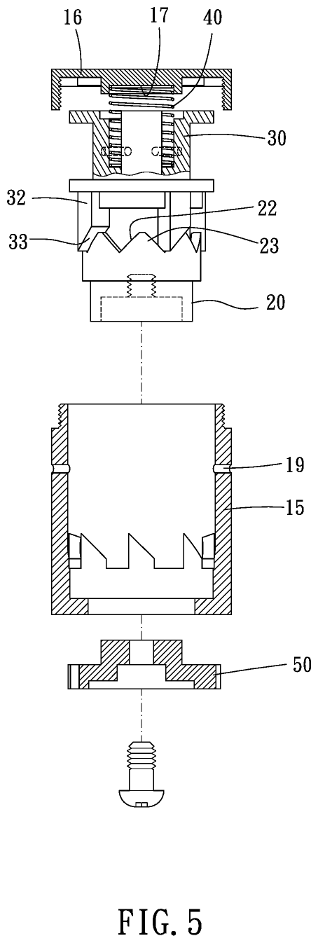

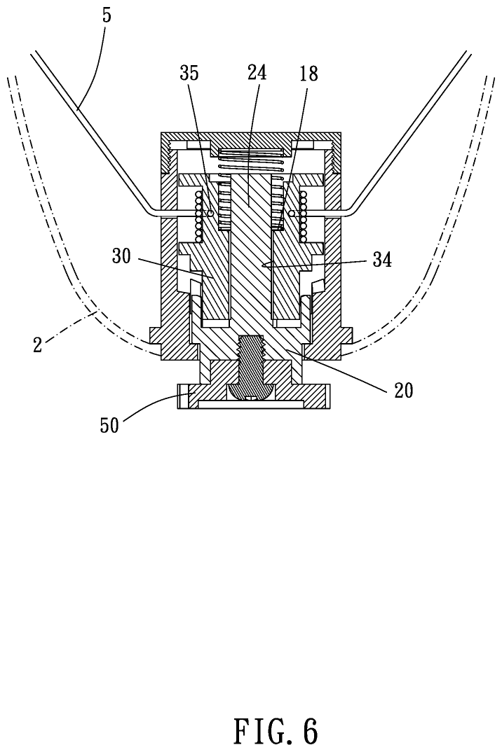

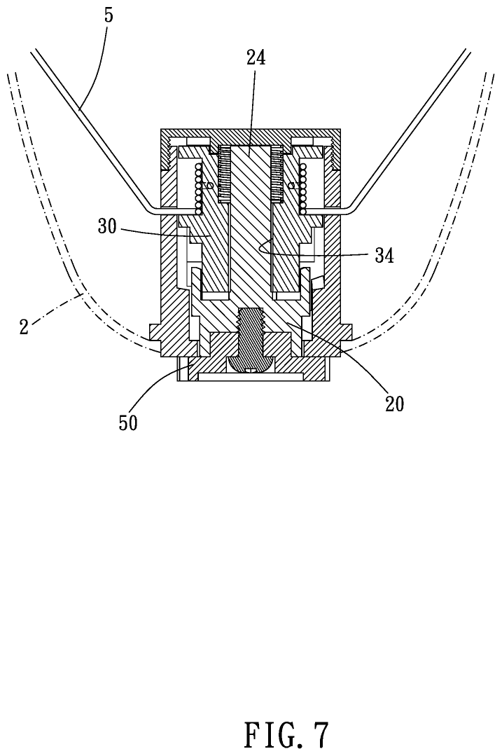

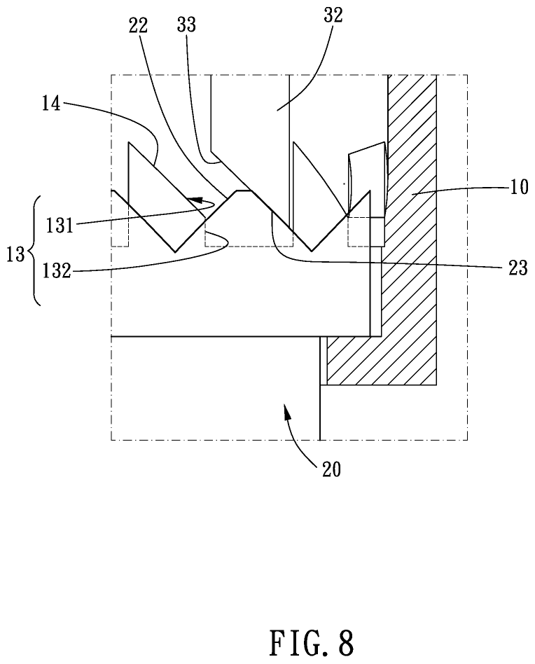

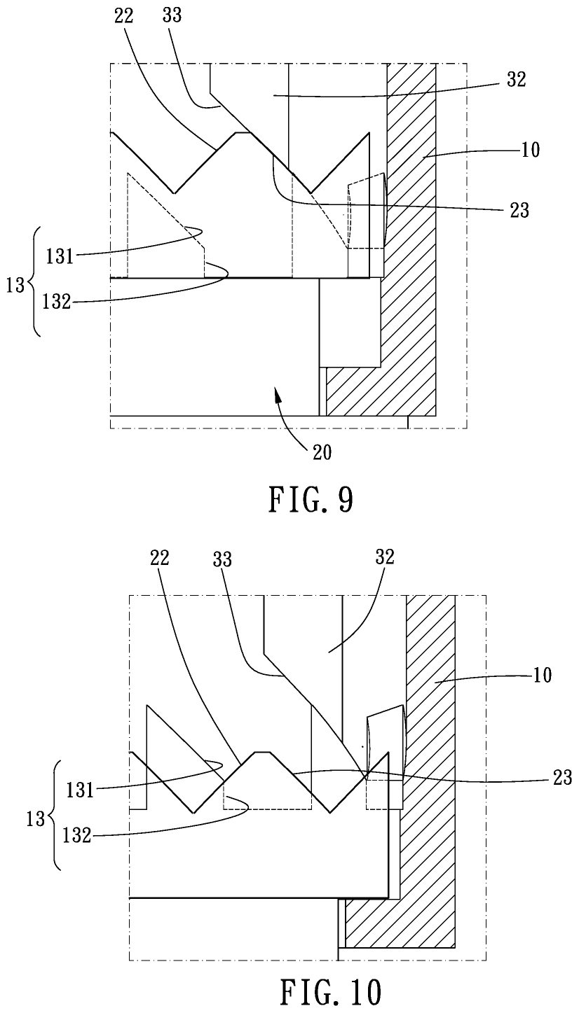

Please refer to FIGS. 1 to 10 for a preferred embodiment of the present invention. An adjustable neck collar includes a reel device 1 and a neck collar main body 2.

The neck collar main body 2 includes a neck sleeve 3, a chin support 4 which is movably connected to the neck sleeve 3, two connecting members 6 and two position-restricting members 7, each of two opposite sides of the neck sleeve 3 has a slant guide slot 8, each of the two connecting members 6 is slidably disposed in one of the slant guide slots 8, and the two position-restricting members 7 are respectively disposed on the two opposite sides of the neck sleeve 3; in other embodiments, the slant guide slot may be disposed perpendicularly.

The reel device 1 is disposed on the neck sleeve 3, the reel device 1 includes a barrel body 10, an axle member 20, a reel member 30, a spring 40 and at least one fastening member 5, a circumferential wall of the barrel body 10 has a first engaging toothed portion 11, the axle member 20 is axially movably pivoted to the barrel body 10, the axle member 20 has a second engaging toothed portion 21, the reel member 30 is axially movably pivoted to the barrel body 10, the reel member 30 has a third engaging toothed portion 31, the third engaging toothed portion 31 is detachably engaged with the first engaging toothed portion 11, the axle member 20 is configured to drive the reel member 30 to axially detach from the first engaging toothed portion 11, the second engaging toothed portion 21 is detachably engaged with the third engaging toothed portion 31, the spring 40 abuts against and between the barrel body 10 and the reel member 30, the at least one fastening member 5 is disposed through the reel member 30, the at least one fastening member 5 is connected to the chin support 4, each of two end portions of the at least one fastening member 5 is movably wrapped on one of the two position-restricting members 7 and connected to one of the two connecting members 6, and each of the two position-restricting members 7 is provided for each of the at least one fastening members 5 to wrap thereon and change direction. The reel device 1 has a simple structure and is convenient for releasing and wrapping a line. In this embodiment, the spring 40 is a compression spring or an expansion spring so that the third engaging toothed portion 31 can be engaged with the first engaging toothed portion 11 normally to prevent the reel member 30 from rotating relative to the barrel body 10.

The barrel body 10 has a through hole 12, the first engaging toothed portion 11 is circumferentially disposed on a hole wall of the through hole 12, the axle member 20 is disposed through the through hole 12, and the reel member 30 is received in the through hole 12 to stably operate. In other embodiments, the first engaging toothed portion may be circumferentially disposed on a circumference of the barrel body. In this embodiment, the first engaging toothed portion 11 is integrally formed on the barrel body 10; and the first engaging toothed portion 11 may also be fixed on the barrel body 10.

Specifically, the second engaging toothed portion 21 has a plurality of first inclined faces 22 circumferentially arranged, the third engaging toothed portion 31 includes at least one tooth block 32, in this embodiment, the at least one tooth block 32 is plural, each of the at least one tooth block 32 has a second inclined face 33, the first engaging toothed portion 11 includes a plurality of tooth grooves 13, each of the plurality of first inclined faces 22 corresponds to one of the plurality of tooth grooves 13 and one of the at least one second inclined face 33 axially, the at least one tooth block 32 is detachably engaged with one of the plurality of tooth grooves 13, and each of the plurality of first inclined faces 22 is configured to push one of the at least one second inclined face 33 to make the at least one tooth block 32 detach from the first engaging toothed portion 11 and guide each of the at least one tooth block 32 to move one of the plurality of first inclined faces 22 to another one of the plurality of tooth grooves 13 so as to restrict or allow the reel member 30 to rotate relative to the barrel body 10. Preferably, each of the plurality of tooth grooves 13 has a slant guide wall 14 for the at least one tooth block 32 to be smoothly engaged with one of the plurality of tooth grooves 13. In addition, each of the at least one second inclined face 33 and the slant guide wall 14 of each of the plurality of tooth grooves 13 are substantially the same in gradient so as to smoothly and stably slide relative to each other. The second engaging toothed portion 21 includes a plurality of apical teeth 23 which are continuously arranged next to each other, and each of the plurality of apical teeth 23 has one of the plurality of first inclined faces 22 to continuously control the reel member 30 to rotate. In other embodiments, the third engaging toothed portion has a plurality of tooth blocks which are continuously arranged next to each other. In addition, the apical tooth may be an L-shaped tooth, T-shaped tooth or tooth members in other shapes for axially and laterally pushing the third engaging toothed portion.

Specifically, each of the plurality of tooth grooves 13 includes a radially-widen section 131 and a radially-narrowed section 132 to stably position. Each of the at least tooth block 32 is smaller than or equal to the radially-narrowed section 132 in width.

One of the axle member 20 and the reel member 30 has an axial hole, and the other of the axle member 20 and the reel member 30 has an axial column which is movably disposed within the axial hole. In this embodiment, the reel member 30 has an axial hole 34, the axle member 20 has an axial column 24 which is movably disposed within the axial hole 34; preferably, the axial column 24 is disposed through the spring 40 so that the spring 40 will not deviate easily during a movement process.

The barrel body 10 includes a main body 15 and a cover body 16, the first engaging toothed portion 11 is disposed in the main body 15, and the cover body 16 is detachably connected to the main body 15; in this embodiment, the cover body 16 is screwed to the main body 15. The cover body 16 may also be clamp, stuck or pivoted to the main body 15. Preferably, the cover body 16 has a groove 17, one of two ends of the spring 40 is received within the axial hole 34 and abuttable against a stepped edge 18 of the axial hole 34, and the other of the two ends of the spring 40 is received within the groove 17 so as to positioningly restrict the spring 40. In other embodiments, the cover body has a protrusion, and the spring is sleeved on the protrusion.

In addition, the reel member 30 has at least one line penetrating hole 35, and the at least one fastening member 5 is disposed through the at least one line penetrating hole 35, and specifically, each of the at least one line penetrating hole 35 includes two openings 36 which are communicated with each other; therefore, the at least one fastening member 5 can be disposed through one of the two openings 36 into the at least one line penetrating hole 35 and out of the other of the two openings 36 so as to connect the reel member 30. In this embodiment, a number of the at least one line penetrating hole 35 is two, a number of the at least one fastening member 5 is two, the two line penetrating holes 35 is disposed on two opposite sides of the reel member 30 and open radially, and the two fastening members 5 are disposed through the two line penetrating holes 35. Furthermore, the barrel body 10 has at least one penetrating hole 19 radially, in this embodiment, the at least one penetrating hole 19 is plural, and the at least one fastening member 5 is disposed through each of the at least one penetrating hole 19.

The adjustable neck collar further has a knob 50, and the knob 50 is detachably disposed to the axle member 20, in this embodiment, the knob 50 is screwed to the axle member 20 to be quickly assembled or disassembled. In other embodiments, the knob may be engaged or clamp to the axle member.

In practice, a user needs to press the knob 50 to drive the axle member 20 to axially move relative to the barrel body 10 to abut against and push the reel member 30 to move axially so that the third engaging toothed portion 31 can be detached from the first engaging toothed portion 11 and clockwise rotates the axle member 20 to make the second engaging toothed portion 21 to be engaged with the third engaging toothed portion 31, each of the at least one fastening member 5 is wrapped in the reel device, each of the two connecting members 6 moves forward along one of the two slat guide slots 8 to adjust the chin support 4 to a higher position relative to the neck sleeve 3. When reaching a position needed, the user can release the knob 50, the axle member 20 and the reel member 30 elastically recover to the original positions, and the third engaging toothed portion 31 is engaged with the first engaging toothed portion 11 again and unable to rotate. In addition, if the user wants to release each of the at least one fastening member 5, s/he needs to press the knob 50 to drive the axle member 20 and the reel member 30 to rotate counterclockwise to release the line, and each of the two connecting members 6 moves backward along one of the two slat guide slots 8 to adjust the chin support 4 to a lower position relative to the neck sleeve 3. If the user wants to slightly adjust the position of the chin support 4, s/he only needs to press the knob 50 to drive the axle member 20.

While we have shown and described various embodiments in accordance with the present invention, it should be clear to those skilled in the art that further embodiments may be made without departing from the scope of the present invention.

What is claimed is:

1. An adjustable neck collar, including:
a neck collar main body, including a neck sleeve, a chin support which is movably connected to the neck sleeve, two connecting members and two position-restricting members, each of two opposite sides of the neck sleeve having a slant guide slot, each of the two connecting members being slidably disposed in one of the slant guide slots, the two position-restricting members being respectively disposed on the two opposite sides of the neck sleeve;
a reel device, disposed on the neck sleeve, the reel device including a barrel body, an axle member, a reel member, a spring and at least one fastening member, a circumferential wall of the barrel body having a first engaging toothed portion, the axle member being axially movably pivoted to the barrel body, the axle member having a second engaging toothed portion, the reel member being axially movably pivoted to the barrel body, the reel member having a third engaging toothed portion, the third engaging toothed portion being detachably engaged with the first engaging toothed portion, the axle member being configured to drive the reel member to axially detach from the first engaging toothed portion, the second engaging toothed portion being detachably engaged with the third engaging toothed portion, the spring abutting against and between the barrel body and the reel member, the at least one fastening member being disposed through the reel member, the at least one fastening member being connected to the chin support, each of two end portions of the at least one fastening member being movably wrapped on one of the two position-restricting members and connected to one of the two connecting members.

2. The adjustable neck collar of claim 1, wherein the second engaging toothed portion has a plurality of first inclined faces circumferentially arranged, the third engaging toothed portion includes at least one tooth block, each of the at least one tooth block has a second inclined face, the first engaging toothed portion includes a plurality of tooth grooves, each of the plurality of first inclined faces axially corresponds to one of the plurality of tooth grooves and one of the at least one second inclined face, the at least one tooth block is detachably engaged with one of the plurality of tooth grooves, and each of the plurality of first inclined faces is configured to push one of the at least one second inclined face.

3. The adjustable neck collar of claim 2, wherein each of the plurality of tooth grooves has a slant guide wall.

4. The adjustable neck collar of claim 1, wherein the second engaging toothed portion includes a plurality of apical teeth which are continuously arranged next to each other, and each of the plurality of apical teeth has a first inclined face.

5. The adjustable neck collar of claim 1, wherein the first engaging toothed portion includes a plurality of tooth grooves, each of the plurality of tooth grooves includes a radially-widen section and a radially-narrowed section, the third engaging toothed portion includes at least one tooth block, and each of the at least one tooth block is engageable with one of the plurality of tooth grooves.

6. The adjustable neck collar of claim 1, wherein one of the axle member and the reel member has an axial hole, and the other of the axle member and the reel member has an axial column which is movably disposed within the axial hole.

7. The adjustable neck collar of claim 1, wherein the barrel body includes a main body and a cover body, the first engaging toothed portion is disposed in the main body, and the cover body is detachably connected to the main body.

8. The adjustable neck collar of claim 1, wherein the reel member has at least one line penetrating hole, and the at least one fastening member is disposed through the at least one line penetrating hole.

9. The adjustable neck collar of claim 8, wherein each of the at least one line penetrating hole includes two openings which are communicated with each other.

10. The adjustable neck collar of claim 3, wherein the barrel body has a through hole, the first engaging toothed portion is circumferentially disposed on a hole wall of the through hole, the axle member is disposed through the through hole, and the reel member is received in the through hole; each of the at least one second inclined face and the slant guide wall of each of the plurality of tooth grooves are substantially the same in gradient; each of the plurality of tooth grooves includes a radially-widen section and a radially-narrowed section, the third engaging toothed portion includes at least one tooth block, each of the at least one tooth block is smaller than or equal to the radially-widen section in width, and each of the at least one tooth block is engageable with one of the plurality of tooth grooves; the at least one tooth block is plural; the reel member has an axial hole, and the axial member has an axial column which is movably disposed within the axial hole; the axial column is disposed through the spring; the barrel body includes a main body and a cover body, the first engaging toothed portion is disposed in the main body, and the cover body is detachably connected to the main body; the cover body is screwed to the main body; the cover body has a groove, one of two ends of the spring is received within the axial hole and abuttable against a stepped edge of the axial hole, and the other of the two ends of the spring is received within the groove; the reel member has at least one line penetrating hole, and the at least one fastening member is disposed through the at least one line penetrating hole; each of the at least one line penetrating hole includes two openings which are communicated with each other; the adjustable neck collar further includes a knob, and the knob is detachably disposed to the axle member; the barrel body has at least one penetrating hole radially; the second engaging toothed portion includes a plurality of apical teeth which are continuously arranged next to each other, and each of the plurality of apical teeth has one of the plurality of first inclined faces.
